(19) United States
(12) Patent Application Publication
(10) Pub. No.: US 2011/0177550 A1
(43) Pub. Date: Jul. 21, 2011

(54) PROCESS FOR THE TREATMENT OF WATER AND PRODUCTION OF BIOMASS AND ASSOCIATED SYSTEMS

(75) Inventor: David McMurran, Cherrybrook (AU)

(73) Assignee: BRINEMAG PTY LTD, Cherrybrook (AU)

(21) Appl. No.: 13/001,957

(22) PCT Filed: Aug. 28, 2009
(86) PCT No.: PCT/AU2009/001119

§ 371 Date: Apr. 4, 2011

(30) Foreign Application Priority Data
Jun. 30, 2008 (AU) 2008903353

(51) Int. Cl. C12P 1/00; C12M 1/00; C02F 1/461; C02F 1/469

(52) U.S. Cl. 435/41; 435/289.1; 204/518; 210/153; 205/742; 204/194

(57) ABSTRACT

A process for the treatment of a saline water including: treating the water to adjust the salinity thereof and produce treated water having a predetermined salinity level; and directing at least a portion of the treated water having said predetermined salinity level to a bioreactor housing a microalgae for generating biomass; wherein said predetermined salinity level of the treated water is predicated by the specie of microalgae housed in the bioreactor.

### FIELD OF THE INVENTION

The invention relates generally to a process for the treatment of water and production of biomass and to associated systems. In certain embodiments, the invention provides for the treatment and application of water in the production of biofuel using bioreactors and/or ponds housing microalgae. In other embodiments, the invention provides for the treatment and application of water in agriculture and forestry, particularly the farming of saline tolerant plants.

### BACKGROUND TO THE INVENTION

Water supply is increasingly becoming an issue around the world, particularly due to global climate change, water usage and pollution. Aquifers have been used to excess and in some countries have been depleted by industry and human consumption beyond recovery levels given rainfall patterns predicted by climate change scientists and taking into consideration the natural expansion of human populations.

Rivers are becoming polluted from human activities associated with industry, agriculture and waste management. Also, chemical applications to improve pasture yields for food crops have caused salinity levels to rise in many regions.

Many governments are facing restricted water supply issues for all human activities including human consumption, industrial and agricultural applications.

To alleviate current and future water supply issues, the desalination of water is being applied widely. Desalination is being employed in coastal areas using seawater desalination technologies. Inland regions where coastal water is not available may employ reverse osmosis (RO) for the desalination of river water, ground water and aquifer water. This delivers a reject stream of bulk concentrated bitterns that may leach into ground water over time if not treated. In some regional centres recycling of human waste water occurs for human consumption and agriculture.

All of these sources of water demonstrate the need to recover reject streams to avoid further damage to the environment. A by-product of desalination, whether located coastally or at inland locations, is the generation of waste salts that are either sent to ocean outfall or accumulated in evaporative ponds. A problem with ocean discharge is the environmental damage to marine life and plants and the potential acidification of ocean water around major cities. This may affect fishing and the ability of the ocean to absorb additional carbon dioxide.

Likewise, the use of evaporative inland ponds may eventually result in the formation of salt encrusted areas that with river flooding or exceptional rainfall may leach salts into the general environment, thereby spreading dry land salinity. Many inland areas, particularly in dry countries like Australia, are already salt affected and ground water salinity has become an increasing ecological problem.

The acidification of seawater in bays or harbours reduces oxygen levels in the water. This has adverse effects on phytoplankton whose survival is at risk from high levels of carbon dioxide in seawater. Phytoplankton, in the form of microscopic life forms, is the principal feedstock of krill which is the main food source for many fish and, in particular, whales. As such, the survival of phytoplankton is vital to the ecological stability of the ocean environment.

Likewise, pteropods may be affected by high seawater acidity. Pteropods are microcellular organisms that have protective shells derived from magnesium and calcium, but particularly calcium, in the surrounding seawater. Lower pH or high seawater acidity adversely affects the growth and thickness of the protective shell. This in turn results in the pteropods becoming fragile and more to susceptible to the sea environment.

The present invention advantageously provides a process that reduces the environmental impact of waste water, such as that generated from desalination operations. The invention also advantageously provides a means of economically using high salinity water obtained from other sources. Each application generally involves the integration of a recovery process with downstream application of the minerals recovered. Peripheral processes may also be provided that enhance the commercial and environmental advantages of the invention.

### SUMMARY OF THE INVENTION

According to one aspect of the invention there is provided a process for the treatment of a saline water including:
treating the water to adjust the salinity thereof and produce treated water having a predetermined salinity level; and
directing at least a portion of the treated water having the predetermined salinity level to a bioreactor housing a microalgae for generating biomass;
wherein the predetermined salinity level of the treated water is predicated by the specie of microalgae housed in the bioreactor.

As used herein, the term “bioreactor” includes within its scope a pond, particularly a closed pond. The term also includes a bioreactor module for initial growth of algae in combination with a pond for cultivation and harvesting of the algae. The term should not be construed as being solely limited to a modular bioreactor unit.

The method of treating the water is not particularly limited, as will be discussed in more detail below. Preferably, the treatment of the water includes electrodialysis of the water to provide the predetermined salinity level.

In certain embodiments it may be desirable to include a plurality of bioreactors are provided, each housing a different species of microalgae. In that case, the treatment of the water may include electrodialysis or capacitive deionisation to provide a plurality of outlet streams, each independently having a predetermined salinity level predicated by the specie of microalgae housed in the bioreactor to which each respective outlet stream is reported. The different species of microalgae housed in the bioreactors may, for example, produce different fuel products.

In order to assist growth of the algae, a nutrient containing waste water, such as from municipal waste, may be introduced to the bioreactor to provide additional nutrient to the microalgae.

Likewise, as will be discussed in more detail below, carbon dioxide may be injected into the treated water that is directed into the bioreactor may be injected directly into the bioreactor. This may achieve desirable carbon dioxide levels for algae growth and/or harvest.

According to another aspect of the invention there is provided a process for the production of a biomass including:
feeding a saline water to a treatment unit;
treating the saline water to adjust the salinity thereof and produce treated water having a predetermined salinity level;
directing at least a portion of the treated water having the predetermined salinity level to a bioreactor housing a microalgae for generating biomass, wherein the predetermined salinity level of the treated water is predicated by the specie of microalgae housed in the bioreactor; and
collecting the biomass from the bioreactor.

Once again, in a preferred embodiment a nutrient containing waste water, such as from municipal waste, is introduced to the bioreactor to provide additional nutrient to the microalgae. Also, carbon dioxide may be injected into the treated water that is directed into the bioreactor may be injected directly into the bioreactor.

According to a further aspect of the invention there is provided a system for producing biomass including:
a treatment unit for adjusting the salinity of a saline water feed to produce treated water having a predetermined salinity level;
at least one bioreactor in fluid communication with the treatment unit and housing a microalgae for generating biomass, wherein the predetermined salinity level of the treated water is predicated by the specie of microalgae housed in the bioreactor; and
a collector for harvesting and collecting the biomass generated in the at least one bioreactor.

As will be appreciated from the above and following discussions, the system may include a plurality of bioreactors housing different species of microalgae, each bioreactor being in fluid communication with the treatment unit, wherein the treatment unit provides a plurality of outlet streams, each independently having a predetermined salinity level predicated by the specie of microalgae housed in the bioreactor to which each respective outlet stream is reported. As will be appreciated, the bioreactor, or one or more bioreactor, may include a modular bioreactor in combination with an enclosed pond.

The treatment unit may be a single unit, or may include a plurality of units that independently treat a feed of saline water to produce desired outlet streams. Generally, the treatment unit includes at least one electrolysis unit and/or at least one capacitive deionisation water demineralisation (CDWD) unit.

In certain embodiments, the system includes an external engine for pumping the saline water feed and/or the treated water within the system. The system may further include a CO₂ capture unit for capturing CO₂ produced by the external engine, the CO₂ capture unit being in fluid communication with the treated water and/or the or each bioreactor such that CO₂ produced by the external engine can be introduced to the treated water and/or the or each bioreactor.

Referring now to each of the aspects of the invention described above, further details will be provided exemplifying various other embodiments of the invention. For convenience, reference hereafter will be made to “bioreactors”. It is reiterated that this term should be considered to include ponds that house the algae, and combinations of bioreactors and ponds. In that regard, it is generally suggested that a stronger species and one that is less susceptible to infection and contamination from environmental effects may be initially produced in bioreactors. Ponds, however, are considered to be more economic at this time. More particularly, 3.6 Ha of modular bioreactor production will equate to about 3 Ha of pond production. Research and mass balance work indicates modular bioreactors develop yields of 7 kg/m3 and ponds 4 kg/m3. However, capital expenditure for modular bioreactors is about 10 times that of ponds. This makes the economics of modular bioreactors less commercial at current diesel prices. It is anticipated that this gap will close in years to come due to more effective and cheaper modular bioreactor design and/or increased diesel prices.

The water to be treated may be recovered from municipal re-use plants in regional and city locations and may also include the waste stream of concentrated bitterns recovered from reverse osmosis desalination. It is also envisaged that the water may be recovered from drilling operations for coal seam methane gas, in which case CO₂ released during the operations may be utilised for algae growth, as discussed in more detail below. The adjustment of the salinity level advantageously provides the appropriate optimum growth conditions for the microalgae species in question.

The treatment of the water may include subjecting the water to electrodialysis to adjust the salinity of the water to said predetermined salinity level. More particularly, sodium chloride is preferably first extracted from the water using an electrodialysis process. Accordingly, the sodium chloride is selectively pulled from the water by electrically charged cation membranes. Selective separation of the remaining minerals takes place in a secondary bipolar arrangement of a traditional multi-layered electrodialysis stack process (for example as supplied by Tokuyama Corp and Asahi Kasei joint venture, Eurodia, sold under the name BP-E1).

This membrane process provides a multi cellular electrodialysis stack system that will separate sodium, magnesium and calcium by selective electrical attraction of the different valencies of the cations and anions in the solution. That is, the electrodialysis effectively separates minerals of different divalent strength in the electrodialysis stack arrangement.

The capture of various components is made possible by special membranes which can hold and absorb the different minerals according to selective cation attraction. By allowing different cell selectivity the flows are effectively split into the major mineral components.

One form a treatment thought to be of particular relevance is capacitive deionisation. Capacitive deionisation technology (CDT) or capacitive deionisation water demineralisation (CDWD) may be of use for the treatment of water having relatively low salinity levels, for example groundwater which may have a salinity level of up to about 7,000 ppm. It is envisaged that CDWD may therefore be suitable for treatment of water located in inland regions. In these instances, such treatment is seen as more appropriate than more energy extensive processes, such as reverse osmosis. In some instances, CDWD may be powered by a wind turbine thereby providing a very energy efficient means of treatment of the relatively low salinity water. Flow rates currently available for CDWD are from 1 ML/day to 10 ML/day.

The solution diluate stream resulting from electrodialysis or CDWD carries minerals in suspension and separate known processes may be deployed to extract the minerals. Some portion of the minerals may be retained in solution for further downstream processing of water being delivered to the photo-bioreactor pipelines. Particularly, an amount of the minerals may be reintroduced to the water being fed to the photo-bioreactor pipelines to ensure the feed facilitates growth of the microalgae at their respective saline water specification requirements.

An embodiment of the process route includes the use of photo-bioreactor modular technology connected to waste water pipes of municipal re-use plants and desalination plants. The waste water pipes are linked to photo-bioreactor modules to process different algae species in various water qualities. For example, high nutrient waste water from municipal waste plants may be combined with reject water from municipal plants and reverse osmosis desalination plants to provide different nutrient and salinity levels.

The water from municipal waste water plants carries many nutrients that are useful and some that may be stripped and modified to assist the growth of algae, for example nitrogen, phosphorus and carbon dioxide. Small concentrations of magnesium and calcium may also assist the photosynthesis process when submitted to the algae species selected for the predetermined quality of water.

It is noted that many municipal waste water facilities are not 100% recycling facilities and still produce a waste reject water that would have to be recycled additional times. The rejected waste water is, however, considered suitable for algal growth as it contains nutrient levels that assist such growth.

It is envisaged that the above process may enable treatment of waste water in regional municipal plant locations to form a small local independent source of biodiesel for councils or local co-operatives that may emerge as a result of the predicted peak oil supply problems of petroleum based diesel in regional areas.

Since algae are part of the carbon cycle of the earth and of plant carbon cycle in particular, carbon dioxide forms part of the growth cycle of the algae. Algae take carbon dioxide in as part of the natural process of photosynthesis. Many algal species thrive in environments where CO₂ is fed directly to the water in which the algae live.

Harvesting algae may also be assisted when the algae become stressed. Stress can take place when nutrients are withheld or CO₂ is pressurised. An embodiment of the process of the invention includes delivering CO₂ in the water to stress the algae at the time of harvesting. The use of super critical CO₂ (pressurized) may also enhance the harvest time. It is envisaged that the CO₂ in both normal and scCO₂ forms may be integrated with RO desalination established in an industrial setting where CO₂ can be fed directly by pipeline into the reject streams from the desalination plant.

Likewise, CO₂ may be injected into the salinity adjusted water flowing to the photo-bioreactor. This embodiment again particularly applies to use of CO₂ in an integrated industrial setting where waste CO₂ from an industrial process may be captured and fed to the waste water being fed to the photo-bioreactor. Where industrial waste CO₂ is not available it is envisaged bottled industrial CO₂ may be used. In rural settings an enclosed micro climate greenhouse may be utilized for growing algae in covered open ponds and aquatic species used to produce CO₂ which is captured in the greenhouse and fed back through photosynthesis to the algae. In settings where CO₂ is extracted from a coal seam gas drilling operation, the CO₂ extracted may be used as needed. In these instances, however, there may be pre-treatment required, for example to remove sulfur.

It is further envisaged that there may be potential to operate pumps and cogen engines for pumping water on, for example cotton irrigation pans or other crops using biodiesel made from the algae. As biodiesel is a blended fuel the exhaust from the engine will contain CO, CO₂ and CO₃ but no harmful mineral contaminants. The exhaust if fed back into the solution water in an algae pond or photo-bioreactor arrangement will advantageously deliver sufficient CO₂ to act as a nutrient to the algae. The system is therefore a sustainable loop for CO₂. The engine output is advantageously matched to the pond or photo-bioreactor size so that excess CO₂ is not delivered. A 1200 liter/day photo-bioreactor system will utilize a specific amount of CO₂ corresponding to the exact requirement for sustained algal growth. As such, a specific CO₂ requirement can achieved using a specific output level of an engine for a photo-bioreactor volume or equivalent pond structure. No surplus CO₂ needs to be added to the atmosphere and all CO₂ is consumed in the specific production route thereby making the system an enclosed loop.

The process of the invention may be associated with processes for the desalination of seawater, and in particular reverse osmosis production that typically delivers 50% concentrated waste bitterns from repeated recirculation of the waste brine. The recycling through membranes a number of times lowers risk of organic clogging of membranes and extracts successive levels of the contained salts. When collected for mineral separation the brine may contain salts more highly concentrated than seawater and typically 70,000 ppm.

The process may be commercially deployed due to the high flow rates of RO desalination plants, typically of the order of 150 mega litres/day flow rate. By adjusting the flows of return salinities, streams of waste desalination water can be delivered to species of microalgae thereby achieving faster growth rates and high oil lipid levels in a synthetically created saline water environment.

As previously noted, certain embodiments of the invention include adjusting the feed water to provide a plurality of streams of water having different salinity. Each stream is designed to support different algae species that grow in varying salinity levels. Examples of the species that may be grown differing salinities include Chlorella Spirulina; Dunaliella; Botlyococcus Bruneii; Nanochloropsis and variations of such species.

Differing water qualities are required for each species. Under this invention sodium chloride recovered from the first pass of electrodialysis processing is resubmitted to water from a stage one storage tank. The dosing of recovered sodium chloride into each separate water flow is adjusted for each of the different species of microalgae. The purpose is to provide saline water of very low salinity level adjusted to the specific quality of water required for the individual species of algae.

The photo-bioreactors are generally modular in design. As such, according to the invention the intention is to feed water having varying water qualities and salinity to separate modular units such that the microalgae species selected facilitate recovery of specific grades of algal oil. The chemistry of the algae oil content may deliver products suitable for either biodiesel or ethanol processing applications. In this way, different grades of biomass oil may be produced.

Reverse osmosis desalination has in recent years become the technology of choice for recovering drinking water from seawater due to its low cost of production. Indeed, technological advances have reduced power consumption to levels comparable to those used piping water from dams.

The modular design of the photo-bioreactor also facilitates varying scale of production. To that end, the invention may also be applicable to brackish water typical to inland saline ground water, river water and aquifers.

The mineral separation used on such inland waters can typically employ small scale reverse osmosis units attached to wind turbine pumping systems for driving the pumps. However, more economical methods, such as CDWD as previously discussed, are preferred. In such regions it is considered that in addition to the production of microalgae using saline adjusted water, the diluate stream can supply water to agriculture generally. Hybrid species of poplar trees are grown in saline water up to 7,000 ppm.

Microalgae species such as Chlorella and Spirulina are of particular interest. The Chlorella species may thrive in water having a salinity level of up to 30,000 ppm. Residence time in the bioreactor for this species is generally about 1 to 1.5 weeks.

The Spirulina species may thrive in water having a salinity level of up to 15,000 ppm. Residence time in the bioreactor for this species is generally about one week.

The process in such areas, as a function of economic maximization of land, will deploy saline water capture, using wind turbine power for pumping, and separate the salts by reverse osmosis, or more preferably by CDWD as described above. Water of a quality suitable for both planting saline tolerant poplars and for use in photo-bioreactors for algal oil production may be delivered basically utilizing the same water source.

Processing of water in regions that are inland is advantageously designed to deliver water of suitable quality for algae growth (i.e. to the bioreactors) and an adjusted water of suitable quality for the growth of saline tolerant poplars.

Minerals recovered from the process of the invention include, in large part, magnesia and calcium. Both may stockpiled for combination with forest clippings and trimmings for conversion to a composite material for production of compounds used in skim coatings and wall boards for housing and building applications, or may be otherwise used.

In an alternative embodiment, following anion and cation separation using electrodialysis, a depleted sodium chloride containing stream may be electrolysed to produce sodium hydroxide (NaOH). Addition of carbon dioxide to the sodium hydroxide produces sodium carbonate and calcium is selectively removed from the solution. The residual stream is a concentrated Mg solution. Addition of NaOH results in the precipitation of Mg(OH)₂.

The Mg(OH)₂ may advantageously be employed in a metal forming process involving calcination of the hydroxide to an oxide and subsequent blending with calcium oxide, prior to reduction of the oxide to a magnesium metal vapour which may be subsequently condensed.

The solutions resulting from the above processing may be separated into streams for production of high purity salt by reforming NaOH with HCl to recover additional water.

Sodium carbonate may be recovered as surplus from the CO₂ addition process described above. Also, NaOH may be obtained as a chemical of commercial value to markets.

Due to the flow rates involved in desalination waste streams, however, this reaction alone has been considered appropriate for the growth of microalgae species in photo-bioreactors. As microalgae are known to consume CO₂, and because of the scale of reject water from desalination, large quantities of CO₂ may be absorbed by the algae and provide a social benefit in an integrated industrial setting using CO₂ normally emitted to the atmosphere from adjacent industries.

That is, in dealing with the issue of water treatment from such high flow sources, the biofixation reaction will require a substantial amount of carbon dioxide. Furthermore, the quantity of magnesium hydroxide produced if one were to follow only this route, even though a useful commodity, would be unusable in the growth and harvesting processes of microalgae. Still further, the above reaction generally provides excessive quantities of magnesium hydroxide from the sodium hydroxide precipitation reaction.

Advantageously, certain embodiments of the invention provide for significant energy savings compared with current RO technology. For example, in some embodiments the systems of the invention may have energy consumption of from 20-30%, in some instances as low as 10%, of that seen in conventional RO plants. A further advantage of certain embodiments of the present invention lies in the recovery of up to 80% of the processed water as potable water that may be recycled back to the community for use.

### DETAILED DESCRIPTION OF THE INVENTION

A more detailed description of the invention will now be provided with reference to the accompanying drawings. It will be appreciated that the drawings are provided for exemplification only and should not be construed as limiting on the invention in any way. It will also be appreciated that various side processing options and by-product recirculation routes are not illustrated in the drawings. Such additional options and routes are, however, within the ambit of the present invention. Referring to the drawings:

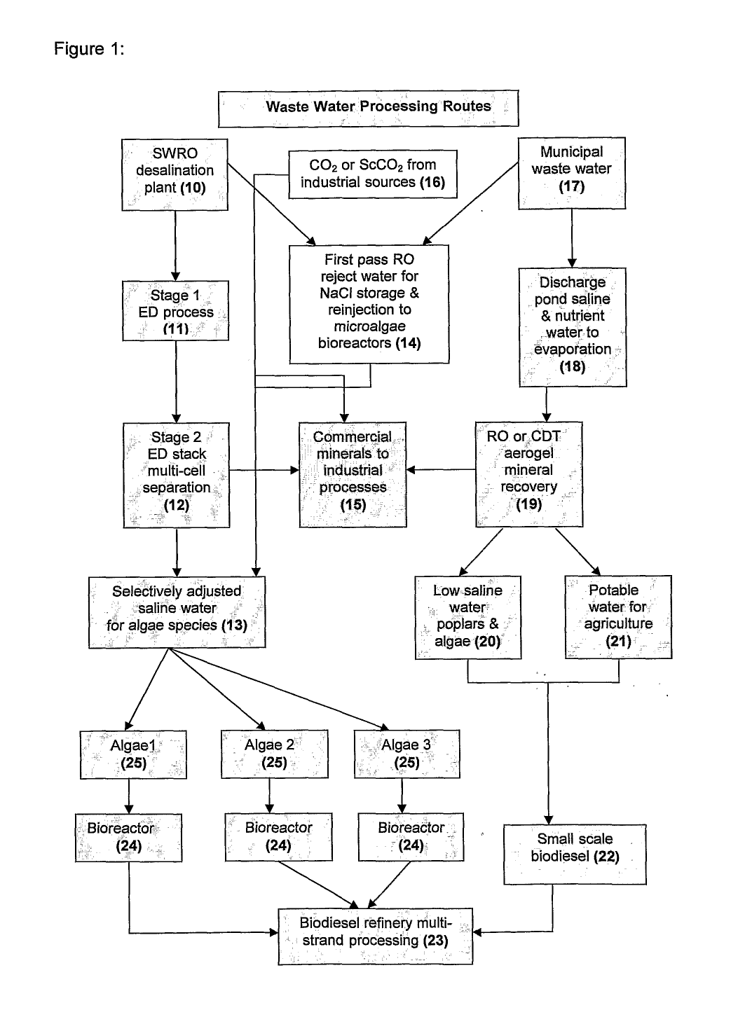

FIG. 1 is a flow chart illustrating a processing route for mineral recovery and saline water quality adjustment for various algae species;

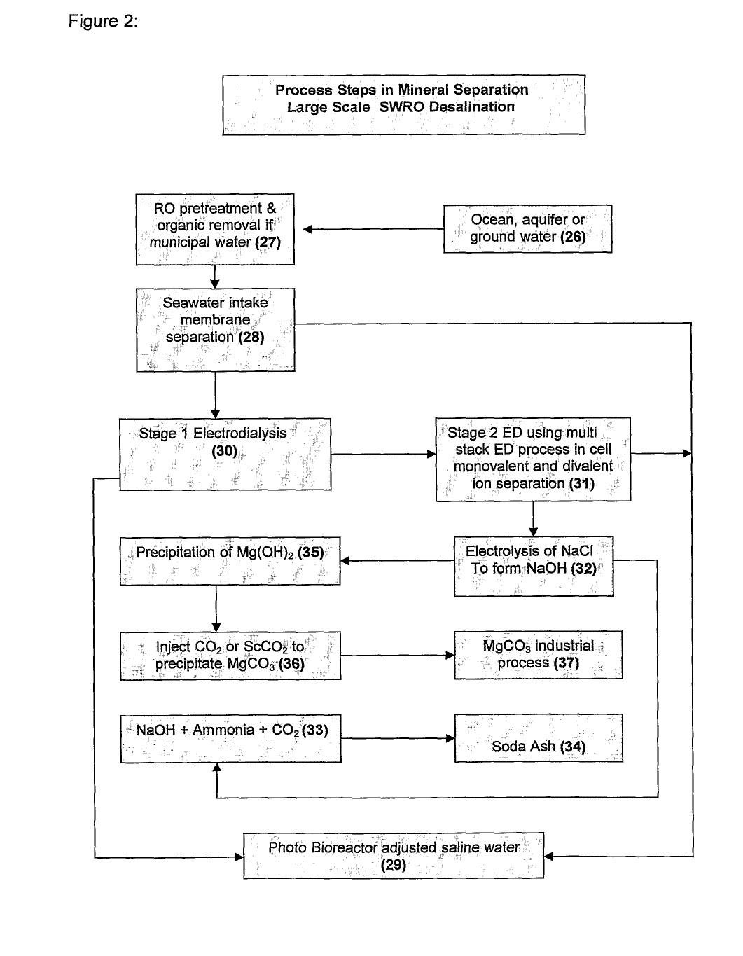

FIG. 2 is a flow chart illustrating a combined processing route for separating minerals from waste streams of a desalination process; and

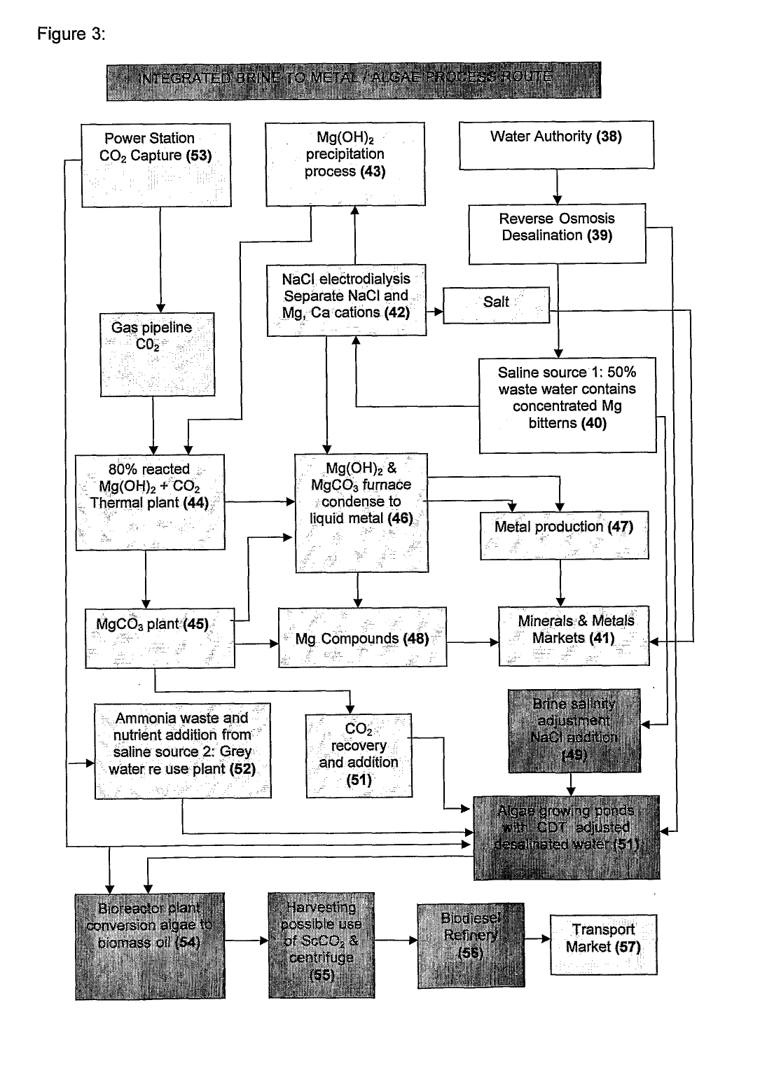

FIG. 3 is flow chart illustrating a combined processing route including sources of components used in the process of the invention and providing a relatively simplified indication of the process steps according to one embodiment of the invention.

Referring to FIG. 1, water from a seawater reverse osmosis (SWRO) plant (10) is passed through a two stage electrodeposition process (11, 12) to produce saline water having an adjusted salinity (13). Reject water from the SWRO plant (10) may be held in storage (14) and re-injected into the water having an adjusted salinity (13) if necessary. Some of the minerals recovered during the two stage electrodeposition process (11, 12) may be utilised commercially (15) as a side product.

Carbon dioxide, as CO₂ gas or ScCO₂ may be supplied from an industrial source (16) and injected into the water having adjusted salinity (13) to provide a desirable level of CO₂ in the water for subsequent use in algae growth and/or harvesting. As previously noted, the CO₂ may also be derived through drilling processes during mining operations or other sources. The CO₂ may also be directed to a recovery process to assist in the recovery of minerals to be used commercially (15).

Municipal waste water (17) may also be employed. Generally, the municipal waste water may be fed to storage (14), or may be discharged to an evaporative pond (18) and subsequently treated (19). Minerals may, again, be recovered and used commercially (15) if desired. Part of the treated waste having low salinity may be fed for use in saline tolerant plant farming or algae growth (20), while potable water recovered may be employed in agriculture (21). This may provide for single species small scale biodiesel production (22) and subsequent biodiesel refining (23).

Once the water having adjusted salinity (13) is at a desired salinity level, CO₂ level and nutrient level, it may be fed to a plurality of bioreactors (24). Again, it should be appreciated that the bioreactors may take the form of ponds, particularly covered ponds. Likewise, the bioreactors may include a combination of a bioreactor and a subsequent pond in combination. Each of the bioreactors (24) houses an algae (25), which may be the same or different. Multiple streams of different water salinity are provided for optimum production of algae in each case. This may advantageously enable some species preferred for ethanol production and some species for biodiesel production to be harvested. For example the Botryococcus species is suitable for ethanol production but has a longer growth time which requires a separated flow from other species selected for biodiesel feedstock. The biodiesel species, in particular Chloralla and Spirulina have a short growth time.

The delivery of different species to modular bioreactors (24) enables treatment of the higher flow rates that are applicable to large SWRO desalination plants.

Turning to FIG. 2, a flowchart exemplifying a mineral separation process from both large scale seawater desalination and a rural setting for a small scale treatment of municipal waste and small scale desalination is provided. A water source (26), which may be derived from the ocean, aquifer, ground water or municipal waste, is fed to a pre-treatment stage (27), which may be reverse osmosis. If municipal waste is involved, the pre-treatment stage (27) may include removal of organic matter.

The pre-treatment stage produces a treated water (28). Pre-treatment may be such that water of a desired salinity is produced, in which case the water may be reported directly to a bioreactor or series of bioreactors (29). Alternatively, or in addition, a stage 1 electrodialysis (30) may be utilised and, also optionally, a stage 2 electrodialysis (31), for example involving a multi-stack electrodialysis. Water of desired salinity may be produced from either stage 1 (30) or stage 2 (31) processing, in which case it may be reported to the bioreactor or series of bioreactors (29).

NaCl separated during the stage 2 (31) processing may be subjected to electrolysis (32) to form NaOH. The formed NaOH may be reacted with ammonia and CO₂ (33) to form soda ash (34). MgOH₂ may also be precipitated (35) and reacted with injected CO₂ and/or ScCO₂ to precipitate MgCO₃ (36). The precipitated MgCO₃ (36) may then be utilised in industrial processes (37).

Referring to FIG. 3, waste derived from coal-fired power plants and desalination plants is substantial, generally due to the extreme throughput of such plants. This presents a globally recognised environmental problem insofar as schedules for the treatment of such high throughput waste streams are relatively difficult to devise. The present invention, at least in certain aspects, aims to utilise carbon dioxide generated during the burning of coal as a feed material to facilitate CO₂ biofixation in algae. The CO₂ may also be used in the recovery of minerals for industrial processing from a high throughput stream derived from desalination or municipal plant settings.

The mineral products separated are commodities that may be put to use in a number of industries, including direct use in the production of biodiesel. As will be appreciated from the above description of the invention, biofuel, such as biodiesel, is a valuable product of the process of the invention.

A feed source (38) feeds a reverse osmosis desalination plant (39). A waste stream (40) from the reverse osmosis desalination plant (39) containing concentrates magnesium bitterns is sourced. Minerals recovered may be utilised in the market (41). The waste stream (40) is treated using electrodialysis (42) to separate sodium chloride from magnesium and calcium cations. Whilst it is not intended to discuss the electrodialysis process in substantial detail here, it is envisaged that this process may advantageously include bipolar membrane electrodialysis. This process, also coined “water splitting”, converts aqueous salt solutions into acids and bases without chemical addition. It is an electrodialysis process since ion exchange membranes are used to separate ionic species in solution with the driving force of an electrical field, but it is different by the unique water splitting capability of the bipolar membrane. In addition, the process offers unique opportunities to directly acidify or basify process streams without adding chemicals, avoiding by-product or waste streams and costly downstream purification steps.

Under the driving force of an electrical field, a bipolar membrane can efficiently dissociate water into hydrogen (H+, in fact “hydronium” H3O+) and hydroxyl (OH−) ions. It is formed of an anion- and a cation-exchange layer that are bound together, either physically or chemically, and a very thin interface where the water diffuses from the outside aqueous salt solutions. The transport out of the membrane of the H+ and OH− ions obtained from the water splitting reaction is possible if the bipolar membrane is oriented correctly (there is no current reversal in water splitting). With the anion-exchange side facing the anode and the cation-exchange side facing the cathode, the hydroxyl anions will be transported across the anion-exchange layer and the hydrogen cations across the cation-exchange layer. Therefore, a bipolar membrane allows the efficient generation and concentration of hydroxyl and hydrogen ions at its surface (up to 10N). These ions are used in an electrodialysis stack to combine with the cations and anions of the salt to produce acids and bases.

A good bipolar membrane has a strong, permanent bond between the two layers and a thin interface to reduce the voltage drop. It also allows the water to easily diffuse inside to the interface and feed the water splitting reaction so that a high current density can be applied to minimize the required membrane area.

Sodium chloride recovered from the electrodialysis process is converted to sodium hydroxide that is used to precipitate magnesium hydroxide in a precipitation process (43). The magnesium hydroxide precipitated may be used as a feedstock for reaction with carbon dioxide (44) to precipitate magnesium carbonate (45). Unreacted magnesium hydroxide may be fed directly to a furnace (46) for reduction to a magnesium metal vapour and subsequent condensing to the liquid metal form (47) that may go to market (41).

If carbon dioxide is used to convert a portion of the magnesium hydroxide to magnesium carbonate, the carbonate form may used as a base stock for the production of magnesium compounds (48) which may be marketed (41). It is also envisaged that in some instances the carbonate form (45) may be directed to the furnace (46), again for reduction and subsequent condensing to liquid magnesium metal (47).

An integrated biodiesel production route is also illustrated in FIG. 3. Several by-products from the integrated process may advantageously be employed providing synergies that result in substantial economic and environmental benefits.

In particular, sodium chloride sourced from the original waste stream is advantageously used as a feed (49) for algae growing ponds (50) containing microalgae. The salinity of the feed may be adjusted as desired depending on the nature of the microalgae being used. Likewise, carbon dioxide (51) recovered from the process in various manners may be fed to the growing ponds as desired, as may waste and nutrients (52) recovered in cases where carbon dioxide is captured from a power station (53) and treated.

Turning to the biodiesel recovery process, microalgae is advantageously transferred to a photo bioreactor plant (54) where it is used to form biomass oil. Microalgae is subsequently harvested (55), possibly using super critical carbon dioxide, which may also be sourced from the fully integrated process, and centrifuging. Biodiesel may be recovered (56) and transported to market (57).

1. A process for the treatment of a saline water including:
treating the water to adjust the salinity thereof and produce treated water having a predetermined salinity level; and
directing at least a portion of the treated water having said predetermined salinity level to a bioreactor housing a microalgae for generating biomass; wherein said predetermined salinity level of the treated water is predicated by the specie of microalgae housed in the bioreactor.

2. A process according to claim 1, wherein the treatment of the water includes electrodialysis of the water to provide said predetermined salinity level.

3. A process according to claim 1, wherein a plurality of bioreactors are provided, each housing a different species of microalgae, and wherein the treatment of the water includes electrodialysis or capacitive deionisation to provide a plurality of outlet streams, each independently having a predetermined salinity level predicated by the specie of microalgae housed in the bioreactor to which each respective outlet stream is reported.

4. A process according to claim 3, wherein the different species of microalgae housed in the bioreactors produce different fuel products.

5. A process according to claim 1, wherein a nutrient containing waste water, such as from municipal waste, is introduced to the bioreactor to provide additional nutrient to the microalgae.

6. A process according to claim 1, wherein carbon dioxide is injected into the treated water that is directed into the bioreactor, and/or is injected directly into the bioreactor.

7. A process according to claim 1 wherein the microalgae is Chlorella species and the treated water has a salinity level of up to 30,000 ppm, and wherein the microalgae is resident in the bioreactor for about 1 to 1.5 weeks.

8. A process according to claim 1, wherein the microalgae is Spirulina species and the treated water has a salinity level of up to 15,000 ppm, and wherein the microalgae is resident in the bioreactor for about one week.

9. A process for the production of a biomass including:
feeding a saline water to a treatment unit;
treating the saline water to adjust the salinity thereof and produce treated water having a predetermined salinity level;
directing at least a portion of the treated water having said predetermined salinity level to a bioreactor housing a microalgae for generating biomass, wherein said predetermined salinity level of the treated water is predicated by the specie of microalgae housed in the bioreactor; and
collecting the biomass from the bioreactor.

10. A process according to claim 9, wherein a nutrient containing waste water, such as from municipal waste, is introduced to the bioreactor to provide additional nutrient to the microalgae.

11. A process according to claim 9, wherein carbon dioxide is injected into the treated water that is directed into the bioreactor and/or is injected directly into the bioreactor.

12. A process according to claim 9, wherein the microalgae is Chlorella species and the treated water has a salinity level of up to 30,000 ppm, and wherein the microalgae is resident in the bioreactor for about 1 to 1.5 weeks.

13. A process according to claim 9, wherein the microalgae is Spirulina species and the treated water has a salinity level of up to 15,000 ppm, and wherein the microalgae is resident in the bioreactor for about one week.

14. A system for producing biomass including:
a treatment unit for adjusting the salinity of a saline water feed to produce treated water having a predetermined salinity level;
at least one bioreactor in fluid communication with the treatment unit and housing a microalgae for generating biomass, wherein said predetermined salinity level of the treated water is predicated by the specie of microalgae housed in the bioreactor; and
a collector for harvesting and collecting the biomass generated in the at least one bioreactor.

15. A system according to claim 14, including a plurality of bioreactors housing different species of microalgae, each bioreactor being in fluid communication with the treatment unit, wherein the treatment unit provides a plurality of outlet streams, each independently having a predetermined salinity level predicated by the specie of microalgae housed in the bioreactor to which each respective outlet stream is reported.

16. A system according to claim 14, wherein the bioreactor or one or more bioreactor includes a modular bioreactor in combination with an enclosed pond.

17. A system according to claim 14, wherein the treatment unit includes at least one electrolysis unit and/or at least one capacitive deionisation unit.

18. A system according to claim 14, including an external engine for pumping the saline water feed and/or the treated water within the system.

19. A system according to claim 18, including a CO₂ capture unit for capturing CO₂ produced by the external engine, the CO₂ capture unit being in fluid communication with the treated water and/or the or each bioreactor such that CO₂ produced by the external engine can be introduced to the treated water and/or the or each bioreactor.
